(19) United States
(12) Patent Application Publication
(10) Pub. No.: US 2004/0175788 A1
(43) Pub. Date: Sep. 9, 2004

(54) Method for the separation of bioproducts

(76) Inventors: Igor Galaev, Lund (SE); Rajni Hatti-Kaul, Lund (SE); Bo Mattiasson, Hjarup (SE)

Correspondence Address: McDermott Will & Emery, 600 13th Street NW, Washington, DC, 20005-3096, US

(21) Appl. No.: 10/481,128

(22) PCT Filed: Jun. 7, 2002
(86) PCT No.: PCT/SE02/01107

(30) Foreign Application Priority Data
Jun. 19, 2001 (SE) 0102171-6

(51) Int. Cl. C12P 21/02; C07K 1/16

(52) U.S. Cl. 435/69.1; 530/412

(57) ABSTRACT

A method for the separation of at least one low molecular weight bioproduct from a cell culture mixture comprising uni-cellular organisms, broth and said at least one bioproduct by passing said cell culture mixture through a bed of an adsorb-ent material to adsorb said at least one bioproduct on said adsorbent material whereas said unicellular organisms and said broth are passing through said bed, whereafter the ad-sorbed bioproduct or bioproducts is/are eluted from said bed of adsorbent material, wherein said adsorbent material on its surface is provided with a material capable of preventing non-specific adsorption of said unicellular organisms to said adsorbent material.

### TECHNICAL FIELD

The present invention relates to a method for the separation of at least one low molecular weight bioproduct from a cell culture mixture and an adsorbent material to be used in said method.

### BACKGROUND ART

A wide variety of low molecular weight products are produced by fermentation processes. The main problem encountered in such processes is their low productivity because of the inhibition or toxicity of the product to the producing microorganisms. A technical solution to overcome this drawback has been to integrate the product recovery step with the fermentation process such that the product is recovered from the fermentation broth while it is being produced (Mattiasson, B. and Holst, O. (1991) Extractive Bioconversions, Marcel Dekker, New York). Such an in situ recovery of the product has also been termed as extractive fermentation. This approach reduces the product concentration to the non-inhibitory levels in the vicinity of the cells, as a result of which the cells convert more substrate to product. Yet another advantage of this technique is that the number of unit operations in the production process is reduced.

The obvious prerequisites for a separation technique to be applicable for extractive fermentation are its ability to handle particulate broth, to be recycled, and be compatible for the producing cells. There are examples given in the literature of extractive fermentation using different separation techniques including membrane filtration, extraction, and adsorption. Adsorption is a mild and robust technique suitable for capture of both low- and high molecular weight products, and when used in a fluidized/expanded bed mode, can be used for processing crude particulate suspensions. One major problem, however, has been the fouling of the adsorbent by non-specific binding of the cells and cell parts. The result is a decrease in adsorption capacity of the resin and also aggregation of the support particles that in severe cases may cause clogging of the column. This problem is significant with ion-exchange adsorbents, which are the workhorse for bioseparations. It is thus of a general interest to find good modes of reducing or preventing cell adsorption to ion exchangers but also to other types of adsorbents.

Accordingly, it is an object of the present invention to provide a method for the separation of at least one low molecular weight bioproduct from a cell culture mixture comprising unicellular organisms, broth and said at least one bioproduct in which method the non-specific adsorption of the unicellular organisms onto an adsorbent used for recovery of said low molecular weight bioproducts is substantially reduced or prevented.

It is another object of the present invention to provide an extractive fermentation method in which the non-specific binding of unicellular organisms to the adsorbent is reduced without affecting its productivity.

It is a further object of the present invention to provide an adsorbent material based on adsorbent material conventionally used in the separation of low molecular weight bioproducts from cell culture mixtures but being modified to exhibit substantially reduced or prevented non-specific adsorption of unicellular organisms thereto.

### BRIEF SUMMARY OF THE INVENTION

The above and other objects are attained by means of the present invention which is based on the discovery that coating the adsorbent material used in the separation of low molecular weight bioproducts from a cell culture mixture with a hydrophilic polymer reduces/prevents non-specific adsorption of the unicellular organisms while still maintaining to a large extent the binding capacity for small molecules. If the adsorbent material is covered with a thin coating of a neutral polymer, the unicellular organisms should be sterically. hindered from gaining access to the surface of the adsorbent material. The small bioproduct molecules, on the other hand, pass through the porous polymer structure and get adsorbed to the adsorbent matrix.

### DETAILED DESCRIPTION OF THE INVENTION

According to one aspect of the invention there is thus provided a method for the separation of at least one low molecular weight bioproduct from a cell culture mixture comprising unicellular organisms, broth and said at least one bioproduct by passing said cell culture mixture through a bed of an adsorbent material to adsorb said at least one bioproduct on said adsorbent material whereas said unicellular organisms and said broth are passing through said bed, whereafter the adsorbed bioproduct or bioproducts is/are eluted from said bed of adsorbent material, wherein said adsorbent material on its surface is provided with a material capable of preventing non-specific adsorption of said unicellular organisms to said adsorbent material.

The term “unicellular organisms” as used here and in the claims includes primarily microorganism cells but also other cells such as mammalian and plant cells are contemplated for use in the method of the present invention.

The term “bioproduct” as used here and in the claims encompasses generally all products obtained by a fermentation process.

The term “low molecular weight” as used here and in the claims is intended to denote a molecular weight of up to 5000 Dalton, preferably of less than 2000 Dalton and most preferably of less than 1000 Dalton.

Low molecular weight bioproducts to be separated in accordance with the method of the present invention are preferably selected from the group consisting of organic acids, organic bases, antibiotic substances and steroids, low molecular weight peptides and amino acids. Particulary preferred bioproducts to be separated by means of the method of this invention are molecular weight hydroxy acids such as lactic acid, amino acids, keto acids and other low molecular weight organic acids, alcohols and aldehydes and among hydrophobic molecules various lipid derivatives.

The adsorbent material to be used in the method according to the present invention may be in practically any shape previously used in conventional separation methods using an adsorbent material for the adsorption of low molecular weight products from solutions or suspensions containing such products. Examples of such shapes are particles, fibres, sheet, membranes and monoliths, the particle form being the preferred shape.

According to one preferred embodiment of the method of the present invention the adsorbent material is an ion exchange material and the material capable of preventing non-specific adsorption of unicellular organisms is a hydrophilic polymer.

Positively as well as negatively charged ion exchange materials can be used in this embodiment. Generally, the prior art ion exchangers conventionally used for the separation of a specific low molecular weight bioproduct from a cell culture mixture can be used for the same purpose in the present invention. Examples of such ion exchange materials include, but are not limited to, DEAE-cellulose, DEAE-Sephadex® and Amberlite® products.

The hydrophilic polymer to be used in this embodiment of the method of the invention may be a non-ionic as well as an ionic polymer.

The non-ionic polymer to be used in this embodiment of the method of the invention may be a polymeric carbohydrate which is selected from the group consisting of agarose, agar, starches, cellulose and (non-ionic) cellulose derivatives or may be a synthetic neutral polymer like polyvinyl alcohol, the preferred non-ionic polymer being agarose.

The ionic polymer to be used in this embodiment of the method of the invention may be selected from the group consisting of polyacryl-derivatives, polyvinyl-derivatives and carbohydrates. Preferably said ionic polymer is selected from the group consisting of poly(acrylic acid), poly(methacrylic acid), poly(styrene sulphonate), poly(ethylene imine), poly(dimethyldiallylammonium)chloride, poly(vinyl pyridine), poly(vinyl amine), poly(allyl amine), chitosan, alginate and dextran sulphate.

In conventional methods using an ion exchange material as the adsorbent with no hydrophilic polymer on its surface there is a substantial degree of non-specific binding of unicellular organisms such as e.g. microorganism cells and cell parts to the adsorbent as previously stated. Such adsorption of cells and cell parts to the ion-exchange matrix (adsorbent) may result in that some of the bound cells and cell parts being eluted with the product that would in turn need further purification. The risks for such events to take place are considerably reduced or even eliminated by means of the hydrophilic polymer coating of the adsorbent material in accordance with the present invention. Any cell adsorbed to the coated adsorbent material may be more easily mobilized as compared to when the cells are bound to the native adsorbent.

In order to improve the stability of the hydrophilic polymer coating said hydrophilic polymer may be cross-linked using cross-linking agents conventionally used for cross-linking of the non-ionic polymer used in each specific case as the coating in accordance with the invention. For instance, agarose may be cross-linked by means of epichlorhydrin, divinyl sulphone and cyanuric chloride. For polymers with carboxylic groups cross-links may be achieved by using hexamethylenediamine and water-soluble carbodiimide.

According to another embodiment of the method according to the invention the adsorbent material is an ion exchange material which is positively charged and the material capable of preventing non-specific adsorption of the unicellular organisms is a negatively charged polymer.

According to a further embodiment of the method of the invention the adsorbent material is an ion exchange material which is negatively charged and the material capable of preventing non-specific adsorption of the unicellular organisms is a positively charged polymer.

According to still another embodiment of the method according to the invention the adsorbent material used is hydrophobic and the at least one low molecular weight bioproduct is a hydrophobic biomolecule binding to said adsorbent material by means of hydrophobic interaction and the material capable of preventing non-specific adsorption of said unicellular organisms is a hydrophilic polymer.

The method according to the invention may be applied to a cell culture mixture obtained from a batch-wise fermentation as well as that-obtained in an extractive fermentation process.

In the method according to the present invention, when applied to an extractive fermentation process, the cell culture mixture from which the low molecular weight bioproduct(s) should be separated is taken from a fermentor and after having passed through the bed of an adsorbent material at least part of the unicellular organisms and the broth is returned to the fermentor.

In this application of the method according to the invention fermentation is preferably carried out using a bed of unicellular organisms growing on a solid porous carrier in a column through which liquid is pumped, which liquid after leaving the column as a cell culture mixture, comprising unicellular organisms, broth and at least one bioproduct to be separated, is passed through the bed of an adsorbent material.

Further according to the invention the bed of an adsorbent material may be packed in a column and the cell culture mixture is caused to flow from the bottom of said column to the top thereof through the bed. The flow of the cell culture mixture through the bed of adsorbent material may be such as to cause the bed of adsorbent material to fluidize/expand.

According to another aspect of the invention there is provided an adsorbent material to be used in the method according to the invention, which adsorbent material comprises a base material which is a conventional ion exchange material or a hydrophobic material conventionally used in separation processes based on ionic and hydrophobic interaction, respectively, and which base material is provided on its surface with a hydrophilic polymer, which may be non-ionic or ionic.

The non-ionic polymer to be applied on the surface of the absorbent base material may be selected from the group consisting of agarose, agar, starches, cellulose and (non-ionic) cellulose derivatives. The preferred non-ionic polymer is agarose.

The ionic polymer to be applied on the surface of the adsorbent base material may be selected from the group consisting of poly(acrylic acid), poly(methacrylic acid), poly(styrene sulphonate), poly(ethylene imine), poly(dimethylallylammonium) chloride, poly(vinyl pyridine), poly(vinyl amine), poly(allylamine), chitosan, alginate and dextran sulphate. The preferred ionic polymer is poly(acrylic acid).

The hydrophilic polymer may be cross-linked after its application on the surface of the adsorbent base material. For this cross-linking those cross-linking agents conventionally used for the cross-linking of the hydrophilic polymer in question are used.

The adsorbent material according to the invention may be prepared by suspending the adsorbent base material in a dilute solution of the hydrophilic polymer, mixing and then draining the solution of the hydrophilic polymer. The coating layer may be from a monolayer thick and thicker. For practical reasons layers of a thickness of more than 50 μm shall be avoided.

The invention will now be further described by means of a number of examples which are not to be construed as limiting the present invention.

### EXAMPLE 1

Materials and Methods

Materials

Amberlite IRA-400 (Röhm and Haas) was procured from ICN (Costa Mesa, Calif.), while agarose (Type IX) was from Sigma (St. Louis, Mo., USA). The remaining chemicals were obtained from standard sources.

Microorganism and Culture Medium

Lactobacillus casei subsp. rhamnosus (DSM 20021) was maintained on MRS-agar (Merck) medium and subcultured fort-nightly. The medium used for lactic acid production contained (per litre) yeast extract, 10 g; K₂HPO₄, 0.5 g; KH₂PO₄, 0.5 g; sodium citrate, 1.0 g; MgSO₄.7H₂O, 0.005 g; MnSO₄.H₂O, 0.0031 g; FeSO₄.H₂O, 0.002 g; and ascorbic acid, 0.005 g; and sugar, 50 g (47.5 g glucose and 2.5 g lactose) or 100 g (95 g glucose and 5 g lactose ). Sterilization of the medium components was performed by autoclaving at 120° C. for 20 min. The stock sugar solution was autoclaved separately prior to mixing with the rest of the medium.

Pretreatment of Ion-Exchange Resin for Lactic Acid Adsorption

Amberlite IRA-400 was regenerated according to the manufacturer recommendations. The resin (200 g) was suspended in deionized water and packed in a water-jacketed column (4×44 cm) and back flushed with deionized water at a flow rate of 10 ml min⁻¹. About 4 bed volumes of 4% (w/v) NaOH were then pumped into the column to replace Cl⁻ ions on the resin with OH⁻ ions, and finally the column was rinsed with deionized water until pH of the eluate was 7.0.

The regenerated resin (200 g) was suspended in 200 ml of 1% (w/v) agarose solution at 45° C., and then mixed gently for 1 h at room temperature (25° C.). Subsequently, the agarose solution was drained, and the resin rinsed with deionized water. The resin was resuspended in deionized water and re-packed in the column, which was allowed to be at 4° C. overnight.

Adsorption of Lactic Acid to Amberlite IRA-400

The column packed with Amberlite IRA-400 (native and agarose coated, respectively) was equilibrated at 42° C. The culture broth (200 ml) obtained at the end of the fermentation containing 84.0 gl⁻¹ of lactic acid and suspended cells (with OD₆₂₀ₙₘ of 6.0) was passed over the Amberlite column at a flow rate of 10 ml min⁻¹ in an upward direction. The column was washed with 300 ml of deionized water, and bound lactic acid was eluted with 300 ml of 2 N HC1. Cell density and lactic acid content in the eluate was determined. The resin was subsequently regenerated for repeated use.

Electron Microscopic Scanning of the Resin

After the culture medium containing the cells and lactic acid was passed through the ion exchange column, the resin was thoroughly washed with deionized water. Samples of resin were taken from the column for electron microscopic studies.

Determination of Lactic Acid Concentration

The total lactic acid content was determined by HPLC (Varian, Calif., USA) using a fermentation monitoring column (150×7.8 mm, Biorad, Hercules, Calif.) and UV detector. The injection volume of the sample was 50 μl. The column, maintained at 65° C., was eluted with 0.014M H₂SO₄ at a flow rate of 0.8 ml min⁻¹ for 20 min. The retention time of lactic acid under these conditions was 5.84 min.

Cell Density Measurement

The density of free cells in the medium was monitored by measuring the absorbance at 620 nm using Shimadzu UV-120-02 spectrophotometer.

Results

Lactic Acid Adsorption from Crude Fermentation Broth

Coating of the adsorbent was performed simply by mixing for some time the resin with the agarose solution at a temperature at which the polymer is still soluble, followed by washing off the excess agarose and storing the resin at a low temperature to stabilize the coating. When the broth containing 84 g 1⁻¹ of lactic acid was passed over an uncoated Amberlite IRA-400 column maintained at 42° C., the adsorption capacity of the resin was about 76 g kg⁻¹. As the ion-exchange column was regenerated and reused, a tendency towards decrease in capacity of the resin was noted. Adsorption to the agarose-coated resin was also done in a similar manner and the resin capacity was seen to be slightly lower, i.e. 72 g kg⁻¹, which, however, remained almost unchanged during repeated use. During this experiment, cell concentration in the flow eluting from the column was also monitored by measuring the absorbance at 620 nm. The reduced optical density of the eluate in case of the uncoated ion-exchanger indicated that the cells are being attached to the resin. Subsequently, the bead surface was observed under electron microscope. Cells were seen to occupy the surface of the native resin but not in case of the agarose coated one.

### EXAMPLE 2

Preparation of PAA-Treated Amberlite IRA-401 and IRA-458

Amberlite IRA-401 (Cl) and IRA-458 (Cl) (BDH, Toronto, Canada) were pretreated according to the manufacturer's recommendations. The resin (6 ml) was suspended in deionized water, packed in a column (15×1 cm I.D.) and washed with deionized water at a linear flow rate of 300 cm/h. About 4 bed volumes of 4% NaOH were then passed through the resin to replace Cl⁻ ions on the resin with OH-groups, and finally the column was rinsed in an upward mode with water until the pH in the effluent was around 7.0. The obtained OH-form of the adsorbent was washed in the same mode with 100 ml of 1% poly(acrylic acid) (PAA) (Fluka, Buchs, Switzerland), pH 9.0 at a linear flow rate of 60 cm/h. Finally PAA-treated Amberlite (PAA-Amberlite) was washed with deionized water in order to remove unbound polyacid.

### EXAMPLE 3

Preparation of Cross-Linked-PAA-Amberlite IRA 458 (cl.-PAA-Amberlite)

Cross-linking of a PAA layer applied on the surface of Amberlite beads was carried out using a procedure including activitation with water soluble carbodiimide, (1-(3-dimethylaminopropyl)-3-ethylcarbodiimide hydrochloride; EDC) (Sigma, St. Louis, Mo., USA) and subsequent treatment with 1,6-hexamethylenediamine (Sigma, St. Louis, Mo., USA) as a cross-linking agent. Amberlite IRA 458 was partially transformed from Cl-form into OH-form using the following procedure: adsorbent (5.5 ml) was suspended in deionized water, placed in the column (15×1 cm I.D.) and washed with 100 ml of deionized water and 20 ml of 0.1% NaOH at a linear flow rate of 300 cm/h with all the washing procedures and pulse injections being performed in an upward mode. After the treatment with NaOH the bed was washed with 30-40 ml of deionized water. Finally 30 ml of solution containing 2% (w/v) PAA (MW 170 000) and 0.2% (w/v) PAA (MW 2 100) was applied to the column at a linear flow rate of 30 cm/h. PAA-coated matrix was washed with water from unbound PAA and transferred to a beaker. The decanted suspension of modified adsorbent was resuspended in 3 ml of deionized water. The solution of carbodiimide (252 mg in 2 ml, pH 6.5-7.0) was slowly added to the gel suspension at constant stirring. The mixture was incubated for 30 min at continuous gentle stirring and adjusting pH to 6.5-7.0 by adding 1 M HCl. After incubation the pH was adjusted to 8.5-9.0 by adding 1 M NaOH and 2 ml of an aquous solution of 1.6-hexamethylenediarnine (15 mg/ml, pH 8.5-9.0) was added. The mixture was incubated for 24 hours at room temperature and 24 hours at 40° C. at continuous shaking. Then the gel was decanted, resuspended in 10 ml of deionized water and put on a shaker for 30 min. The washing procedure was repeated 3 times. Finally the gel was transferred to the column and washed with deionized water containing 0.1 M NaCl until absorbance of the washings at 220 nm was close to zero.

All the experiments described in the following examples were carried out with the OH-form of the native, PAA-(from Example 2) and cl.-PAA-Amberlite (from Example 3). The adsorbent was transformed into OH-form before each chromatographic run by washing the bed with 4 bed volumes of 4 % NaOH and deionized water as described above.

### EXAMPLE 4

Batch Adsorption of Yeast Cells on i) Non-Treated and PAA-Treated Amberlite IRA-401, ii) Non-Treated Amberlite IRA 458 and cl.-PAA-Amberlite IRA 458

In this experiment the following adsorbents were used: i) non-treated Amberlite IRA-401 and Amberlite IRA-401 treated with PAA as described in Example 2, ii) non-treated Amberlite IRA 458 and cross-linked-PAA-Amberlite 458 (cl.-PAA-Amberlite) prepared as described in Example 3.

The adsorbents (0.5 ml of the adsorbent suspended in equal volume of deionized water, pH 7.0) were incubated with 2.5 ml of water containing different concentrations of yeast cells (bakers yeast from a local supplier) (0.8-10.0 mg/ml and 5 0.25-5.00 mg/ml for batch adsorption on Amberlite IRA-401 (i) and Amberlite IRA 458 (ii), respectively), pH 7.0 for 1 hour at room temperature and constant shaking. After incubation on a shaker the adsorbent was settled and the contents of unbound cells in the supernatants were analyzed by measuring absorbance at 620 nm. In the control experiments, 2.5 ml of water, pH 7.0 was used instead of suspensions of Amberlite.

The results thus obtained are reported in the following Table 1.

TABLE 1

| | Bound cells, mg/ml adsorbent | | | |
|---|---|---|---|---|
| | | Amberlite | | |
| Cell | | IRA-401 | | Amberlite |
| concentration | | treated | | IRA-401 treated |
| in suspension | Amberlite | as described | Amberlite | as described |
| mg/ml | IRA-401 | in Ex. 2 | IRA-458 | in Ex. 3 |
| 0.2 | | | 1 | −0 |
| 0.7 | | | 2.5 | 0.1 |
| 1.5 | | | 3.4 | −0 |
| 2 | 7 | 1 | | |
| 2.2 | | | 6 | 1 |
| 2.7 | | | 7 | 1 |
| 3.0 | | | 7.5 | 0.2 |
| 3.3 | | | 7.3 | −0 |
| 5 | 19 | 1.5 | | |
| 10 | 22 | 1 | | |
| 15 | 24 | 1.5 | | |

### EXAMPLE 5

Batch Adsorption of Shikimic Acid on Non-Treated Amberlite IRA 458 and cl.-PAA-Amberlite IRA 458

Non-treated or cl.-PAA-coated Amberlite (prepared as described in Example 3) (0.5 ml of the adsorbent suspended in equal volume of deionized water, pH 7.0) was incubated with 2 ml of water containing different concentrations of shikimic acid (gift from BioGaia Fermentation AB, Lund, Sweden) (2.4-47.0 mg/ml), pH 7.0 for 1.5 hours at room temperature and constant shaking. After the incubation the gel was settled and the content of unbound shikimic acid in the supernatants was analyzed by measuring absorbance at 220 nm. In a control experiment, deionized water was used instead of Amberlite suspension.

The binding of yeast cells to cl.-PAA-Amberlite was considerably reduced as compared to the binding to the non-treated matrix. On the other hand, this batch adsorption experiment demonstrated that the introduction of a cross-linked PAA layer only slightly influenced the capacity of the ion exchanger for the target product, shikimic acid.

### EXAMPLE 6

Fluidized Bed Adsorption of Shikimic Acid and Yeast Cells on Non-Treated Amberlite, PAA- and cl.-PAA-Amberlite

The fluidized mode of adsorption has been used to enable application of cell-containing suspension on an ion-exchange column. Cell-containing suspensions cannot be applied on a packed bed column as the bed presents a depth filter, which retain cells. The cell retention by a packed-bed column resuits in complete blockage of the flow through the column.

The settled bed of adsorbent (non-treated-Amberlite, PAA-Amberlite of Example 2 and cl.-PAA-Amberlite of Example 3) (5-6 ml) in a column (15×1 cm I.D.) was transformed into a fluidized mode by pumping deionized water, pH 7.0 upwards through the bed at a linear flow rate of 610 cm/h. A solution of shikimic acid (10 mg/ml or 20 mg/ml) or a suspension of yeast cells (10 mg/ml) was applied upwards through the bed at the same linear velocity. The content of yeast cells or shikimic acid in the effluent during adsorption, washing and elution stages was analyzed by measuring absorbance at 620 and 230 nm, respectively.

Yeast cells, which were used in this example as model microorganism, are negatively charged at neutral conditions. Hence they have a strong tendency to adsorb to positively charged matrices like Amberlite IRA-401. However, practically no cell binding was observed under the same conditions during incubation of cells with PAA-treated Amberlite, probably due to the cell repulsion by the negatively charged polymer chains.

The fluidization of the Amberlite was achieved at a linear flow rate of 610 cm/h with an 1.8 fold bed expansion. The beds of both non-treated and PAA-coated Amberlite had some back mixing of the adsorbent and represented a mixed mode between completely fluidized bed and expanded bed.

A pulse of cells was passed through fluidized beds of the non-treated and PAA-treated Amberlite IRA-401 in an upward mode. Up to 26% of applied cells were retained by the nontreated matrix and approximately 13% by the PAA-treated matrix. The biomass strongly bound to the non-treated Amberlite and practically was not washed out from the bed during the washing stage. Bound cells caused aggregation of non-treated beads and formation of channels in the fluidized bed was visually observed. However, practically all retained cells were washed out from PAA-Amberlite IRA-401 during the washing stage and no aggregates were observed in this case. Thus, coating of the strong anion-exchange matrix with a layer of PAA proved to be effective against undesirable interaction with the cells.

PAA adsorption to the anion-exchanger results in the occupation of some ion-exchange ligands and hence in a reduced capacity of the matrix towards a low-molecular-weight target subtance, e.g. shikimic acid. To evaluate the significance of the capacity decrease caused by PAA adsorption, the break-through curves for shikimic acid on native and PAA-Amberlite IRA-401 were compared. The close similarity of the break-through profiles for shikimic acid on both adsorbents indicate that the shielding PAA layer was bound mainly at the surface of the beads without affecting much the dynamic binding capacity of the matrix. Probably, the chains of PAA with high molecular weight did not diffuse inside the pores of the beads and thus did not decrease much the total capacity of the adsorbent while effectively shielding the matrix from the interactions with yeast cells.

Cl.-PAA-Amberlite displayed improved flow properties as compared to the unmodified resin. Curves illustrating the dependence of the bed height of cl.-PAA- and non-treated (native) Amberlite on the flow rate of mobile phase were established. Both curves were obtained after the gels were pretreated by washing with 4 bed volumes of 3 M acetic acid followed by washing with deionized water. It must be noted that the pretreatment of the native Amberlite IRA 458 with 3 M acetic acid resulted in the changes in its flow properties. The bed of the native gel pretreated with acetic acid practically did not expand under applied flow as the particles of the resin seemed to be “glued” together. The bed was not fluidized even at flow rates as high as 530 cm/h. The cl.-PAA-Amberlite was, however, easily fluidized. The degree of expansion of the bed increased with the increase in the flow rate. The degree of expansion of 1.8 was achieved at a linear flow rate of 610 cm/h.

Batch adsorption experiments also demonstrated that the introduction of a cross-linked PAA layer only slightly influenced the capacity of the ion exchanger for the target product, shikimic acid: 81.3 and 80.6 mg of shikimic acid bound per ml adsorbent to the native and cl.-PAA-Amberlite, respectively.

The PAA content in shikimic acid preparations eluted from the modified matrix with 3 M acetic acid was below detection limit already after the second elution cycle. Yeast cells applied on a column with a fluidized cl.-PAA-Amberlite bed did not bind to the matrix and were completely washed out with deionized water. The matrix was successfully used in 3 cycles of binding/elution of the shikimic acid in this model system comprising shikimic acid and yeast cells.

### EXAMPLE 7

Adsorption Studies Using Cultur Liquid from Industrial Fermentations

A cell-free fermentation broth from industrial fermentations of shikimic acid was provided by BioGaia AB (Lund, Sweden) and contained about 10 mg/ml shikimic acid. The fermentation broth was applied on a column with freshly prepared cl.-PAA-Amberlite (preparation as in Example 3) (5 ml) in an upward mode at a linear flow rate of 610 cm/h. The column was thoroughly washed with deionized water and the bound shikimic acid was eluted in an upward mode (flow rate of 200 cm/h) with 3 M acetic acid. The resin was regenerated by washing with i) 0.1 M NaCl until the disappearance of adsorbance at 220 nm in the effluent, ii) deionized water, iii) 20 bed volumes of 1% NaOH, iv) deionized water till pH of the effluent was around 7.0. The regenerated column was used either for the next cycle (application of a new portion of the cultur liquid) or for analysis of the interaction of the resin with yeast cells. The analysis of the interaction of cl.-PAA-Amberlite with yeast cells was carried out after the first, the second, the fourth and the fifth cycles. It was carried out as follows: the suspension of yeast cells (30 ml) containing 10 mg/ml cells, pH 7.0-7.5 was applied to the columns in an upward mode at a linear flow rate of 610 cm/h or 300 cm/h (at which only 1.5 times expansion was achieved). The amount of cells washed out from the column was determined spectrophotometrically by measuring absorbance at 620 nm in the effluent.

The capacity of cl.-PAA-Amberlite towards shikimic acid decreased slightly with the increasing numbers of application cycles for the cl.-PAA-Amberlite. Nevertheless the drop. in capacity after five cycles did not reach above 20%. The application of yeast cell suspension did not result in any visible decrease of the cl.-PAA-Amberlite performance and no binding of cells to the cl.-PAA-Amberlite has been detected. Some yeast cells were entrapped in the cl.-PAA-Amberlite bed at lower flow rate of 300 cm/h (1.5 fold bed expansion), but those cells were easily removed by washing at the flow rate of 610 cm/h (1.8 fold bed expansion) used throughout most of the experiments.

However, the application of yeast cells in the column with native Amberlite resulted in the strong binding of about 3 mg of cells per ml of the adsorbent. The performance of the native Amberlite with bound cells was deteriorated even after a rigorous washing procedure including washing with 20 bed volumes of 1% NaOH. Already, after the first cycle, the bed of native Amberlite did not expand any more at the flow rate of 610 cm/h and the further use of the adsorbent was impossible.

1. Method for the separation of at least one low molecular weight bioproduct from a cell culture mixture comprising unicellular organisms, broth and said at least one bioproduct by passing said cell culture mixture through a bed of an adsorbent material to adsorb said at least one bioproduct on said adsorbent material whereas said unicellular organisms and said broth are passing through said bed, whereafter the adsorbed bioproduct or bioproducts is/are eluted from said bed of adsorbent material, wherein said adsorbent material on its surface is provided with a material capable of preventing non-specific adsorption of said unicellular organisms to said adsorbent material.

2. Method according to claim 1, wherein said at least one low molecular weight bioproduct is selected from the group consisting of organic acids, organic bases, antibiotic substances and steroids, low molecular weight peptides and amino acids.

3. Method according to any of claims 1 and 2, wherein said adsorbent material is in the shape of particles, fibres, sheets, membranes or a monolith.

4. Method according to any of claims 1 to 3, wherein said adsorbent material is an ion exchange material and said material capable of preventing non-specific adsorption of said unicellular organisms is a hydrophilic polymer.

5. Method according to claim 4, wherein the hydrophilic polymer is a non-ionic polymer which is a polymeric carbohydrate selected from the group consisting of agarose, agar, starches, cellulose and cellulose derivatives or is a synthetic neutral polymer.

6. Method according to claim 4, wherein the hydrophilic polymer is an ionic polymer selected from the group consisting of polyacryl-derivatives, polyvinyl-derivatives and polymeric carbohydrates.

7. Method according to claim 6, wherein said polyacryl-derivatives, polyvinyl-derivatives and polymeric carbohydrates are selected from the group consisting of poly(acrylic acid), poly(methacrylic acid), poly(styrene sulphonate), poly (ethylene imine), poly (dimethyldiallylammonium) chloride, poly(vinyl pyridine), poly(vinyl amine), poly(allylamine), chitosan, alginate and dextran sulphate.

8. Method according to any of claims 4 to 7, wherein said hydrophilic polymer has been cross-linked after its application on the adsorbent material.

9. Method according to any of claims 1 to 3, wherein said adsorbent material is an ion exchange material which is positively charged and said material capable of preventing nonspecific adsorption of said unicellular organisms is a negatively charged polymer.

10. Method according to any of claims 1 to 3, wherein said adsorbent material is an ion exchange material which is negatively charged and said material capable of preventing nonspecific adsorption of said unicellular organisms is a positively charged polymer.

11. Method according to any of claims 1 to 3, wherein said adsorbent material is hydrophobic and said at least one low molecular weight bioproduct is a hydrophobic biomolecule binding to said adsorbent material by means of hydrophobic interaction and said material capable of preventing nonspecific adsorption of said unicellular organisms is a hydrophilic polymer.

12. Method according to any of claims 1 to 11, wherein said cell culture mixture is taken from a fermentor and after having passed through said bed of an adsorbent material at least part of the unicellular organisms and the broth is returned to the fermentor.

13. Method according to claim 12, wherein fermentation is carried out using a bed of unicellular organisms growing on a solid carrier in a column through which liquid is pumped, which liquid after leaving the column as a cell culture mixture, comprising unicellular organisms, broth and at least one bioproduct to be separated, is passed through said bed of an adsorbent material.

14. Method according to claim 12, wherein said bed of adsorbent material is packed in a column and said cell culture mixture is caused to flow from the bottom of said column to the top thereof through the bed.

15. Method according to claim 14, wherein the flow of said mixture is such as to cause the bed of adsorbent material to fluidize/expand.

16. Adsorbent material for use in the method according to claim 1, which adsorbent material comprises a base material which is a conventional ion exchange material or a hydrophobic material conventionally used in separation processes based on ionic and hydrophobic interaction, respectively, and which base material is provided on its surface with a hydrophilic polymer.

17. Adsorbent material according to claim 16, wherein said hydrophilic polymer is a non-ionic polymer which is a polymeric carbohydrate selected from the group consisting of agarose, agar, starches, cellulose and cellulose derivatives or is a synthetic neutral polymer.

18. Adsorbent material according to claim 16, wherein the hydrophilic polymer is an ionic polymer selected from the group consisting of poly(acrylic acid), poly(methacrylic acid), poly(styrene sulphonate), poly(ethylene imine), poly(dimethyldiallylammonium)chloride, poly(vinyl pyridine), poly (vinyl amine), poly(allylamine), chitosan, alginate and dextran sulphate.

19. Adsorbent material according to any of claims 16 to 18, wherein said hydrophilic polymer has been cross-linked after its application on the adsorbent material.
